(19) United States
(12) Patent Application Publication
(10) Pub. No.: US 2026/0144766 A1
(43) Pub. Date: May 28, 2026

(54) USE OF CANNABINOID 1 RECEPTOR AGONIST ARACHIDONOYL CYCLOPROPYLAMIDE (ACPA) IN NON SMALL CELL LUNG CANCER

(71) Applicants: HACETTEPE UNIVERSITESI, Ankara (TR); ATILIM UNIVERSITESI, Ankara (TR)

(72) Inventors: Ozge BOYACIOGLU, Ankara (TR); Petek KORKUSUZ, Ankara (TR); Elif BILGIC, Ankara (TR); Erem BILENSOY, Ankara (TR); Cem VARAN, Ankara (TR); Emirhan NEMUTLU, Ankara (TR)

(73) Assignees: HACETTEPE UNIVERSITESI, Ankara (TR); ATILIM UNIVERSITESI, Ankara (TR)

(21) Appl. No.: 19/367,872

(22) Filed: Oct. 24, 2025

Related U.S. Application Data
(63) Continuation of application No. 17/617,008.

(30) Foreign Application Priority Data
Aug. 20, 2019 (TR) 2019/12451

(51) Int. Cl. A61K 31/16; A61K 9/51; A61P 35/00

(52) U.S. Cl. CPC A61K 31/16; A61K 9/5153; A61P 35/00

(57) ABSTRACT

The non-small cell lung cancer adenocarcinoma via the pathway mediated with the cannabinoid receptor is targeted for the first time with the synthetic cannabinoid agonist arachidonoyl cyclopropylamide (ACPA). An ACPA-PCL nanoparticle release system based on nanoengineering provides long term-controlled release and stability of the CB1 receptor agonist ACPA following the determination of the efficient dose and the antiproliferative effect of ACPA on the non-small cell lung cancer lines expressing the cannabinoid (CB) receptors in vitro medium at different doses.

### CROSS REFERENCE TO THE RELATED APPLICATIONS

This application is a continuation of U.S. application Ser. No. 17/617,008 filed on Dec. 7, 2021, which is the national phase entry of International Application No. PCT/TR2020/050618, filed on Jul. 13, 2020, which is based upon and claims priority to Turkish Patent Application No. 2019/12451, filed on Aug. 20, 2019, the entire contents of which are incorporated herein by reference.

### TECHNICAL FIELD

The invention is related to the antiproliferative effect of cannabinoid 1 receptor (CB1) agonist Arachidonoyl cyclopropylamide (ACPA) and its release system on non-small cell lung cancer which is late diagnosed and has low clinical treatment efficiency.

### BACKGROUND

Lung cancer, is the most commonly observed malignancy worldwide, having high invasive and metastatic features. Lung cancer is the cancer that has led to the most frequent amount of deaths in the recent years and the five-year survival rate of lung cancer (15.0%) in comparison to other cancers is quite low. Due to high death rates, the primary factors of lung cancer becoming pandemic is that it has become a significant public health issue as it has reached the incidence rate of geographical mortality and that exposure to tobacco smoke has increased. In 2018, over 2 million new lung cancer cases have been defined. 80% of these cases are known to be of non-small cell lung cancer (NSCLC) type.

Various drugs are used that target the tumor in order to treat non-small cell lung cancer, advanced or metastatic non-small cell lung cancer in the prior art. These drugs can be listed as Erlotinib, Gefitinib, Vandetanib, Dacomitinib, Icotinib, Afatinib, Bevacizumab, Crizotinib and Ceritinib. Radiation therapy and surgical treatment is also applied together with chemotherapy depending on the stage of the disease. During meta-analysis total living period with the already applied treatment protocols, survival with non-advancing disease, response rate to treatment and life quality is tried to be developed.

Gefitinib and erlotinib inhibitors for treatment purposes are prevalently preferred in lung cancer patients that express this protein at high levels and that comprise endothelial growth factor receptor (EGFR) mutation. Vandetanib that is used as an oral anticancer agent has an effect specifically over the vascular endothelial growth factor receptor (VEGFR) and EGFR signalling pathway. Dacomitinib has been developed as an EGFR inhibitor. On the other hand the positive effect of the drug Icotinib as an EGFR inhibitor for the treatment of non-small cell lung cancer has been shown in vitro and in vivo studies; and it has been reported that in clinical studies that the drug is effective in the advanced stages of cancer. Afatinib has been used for the treatment of small cell lung cancer since 2013 as an FDA approved drug. It is preferred in patients with EGFR gene mutations or deletions similarly with Erlotinib. Bevacizumab prevents the formation of new blood vessels with VEGF inhibition. Mutation is observed in the anaplastic lymphoma kinase (ALK) and c-ros oncogene 1 (ROS1) genes in some non-small cell lung cancer patients. Accordingly, Crizotinib has been developed as an anti-cancer drug that blocks mutation and stops the growth of the tumor. Crizotinib has been approved by the FDA in the year 2011 as a clinical drug to be used in treating metastatic or advanced stage non-small cell lung cancer. Patients that cannot tolerate Crizotinib or still exhibit an increase in tumor growth although they have been using crizotinib are treated with the anti-cancer drug called Certinib. Certinib is used in metastatic ALK positive lung cancer with a mechanism that is similar to Crizotinib. However, said chemo-therapeutic drugs that are generally systemically applied clinically, for treating non-small cell lung cancer lead to several side effects in patients. Some of the side effects are problems related to memory, fatigue, nausea and vomiting, changes in the musculoskeletal system, hair loss due to weakening of the hair roots and increase in body pain and infection risk.

In the prior art, the apoptotic, antiproliferative, anti-invasive, migration inhibiting, and metastasis and vascularity reducing effects of cannabinoids on various tumor models, have been shown. Cannabinoids show their proapoptotic effects on cells generally via CB1 and/or CB2 receptors which are classical cannabinoid receptors. It has been reported that cannabinoid ligands exhibit proapoptotic effect by inhibiting protein kinase B (Akt) and PI3K via the activation of ceramide synthase or by blocking cyclic adenosine monophosphate (cAMP) pathway through the mediation of CB1/2 receptors. Therefore, proliferation is reduced by the stimulation of the CAMP/protein kinase A/mitogen activated protein kinase (cAMP/PKA/MAPK) pathway, the cell cycle regulator Cdk2 is suppressed and a cyclin-dependent kinase inhibitor p27/kip1 activation is obtained.

The therapeutic effect of cannabinoid receptors CB1 and CB2 against non-small cell lung cancer has been examined in the prior art. Moreover the treatment of non-small cell lung cancer cell lines (A549 and SW-1573) with CB1/CB2 and CB2-specific agonist WIN55,212-2 and JWH-015 has been shown.

The patent document numbered US 2017/0304290 A1 is related with 1-[[[4-(4-fluoro-2-methyl)-1H-indol-5-yl)oxy-6-methoxyquinolin-7-yl]oxy]methyl]cyclopropylamine or pharmacologically acceptable salts thereof that can be use in non-small cell lung cancer treatment.

However none of said documents mention the antiproliferative effect of cannabinoid 1 receptor (CB1) agonist ACPA, on non-small cell lung cancer.

It is known that the CB1 and CB2 receptor levels that are synthesized in non-small cell lung adenocarcinoma tissues are higher than normal lung tissues. The determination of the CB receptor levels in non-small cell lung tumor cells can be as valuable as diagnosis and it may be effective in target-specific treatment.

The cannabinoid 1 receptor is synthesized around 24% in non-small cell lung cancer cases. It has been shown that the metastasis of primary myoblasts to the lung is mediated by the CB1 receptor. Despite this, some research groups have reported that cannabinoids can be effective against lung cancer when mediated with the CB2 receptor. WIN55,212-2 and JWH-133 exogenous cannabinoids exhibit proapoptotic effect by inhibiting protein kinase B (Akt) and PI3K via the activation of ceramide synthase or by blocking cAMP pathway through the mediation of CB1 and/or CB2 receptors in non-small cell lung cancer cells. On the other hand, JWH-133 can prevent invasion by inhibiting matrix metalloproteinase enzymes in non-small cell lung cancer cells. It is known that cannabinoid agonist Δ9-THC prevents the growth of non-small cell lung cancer by means of the inhibition of PI3K/Akt and activation of the ERK1/2 pathway via the mediation of the CB1 and/or CB2 receptors.

In drug therapy, it is crucially important for the drug to reach the targeted area within a certain period of time, at a treatment dose, without the active agent losing its therapeutic effect. The traditional drug formulations that are applied nowadays with medication that has narrow therapeutic index, are inadequate for drug treatment. One other important problem in traditional drug formulations is that the active agent is delivered to areas other than the target area in the body and that is causes undesirable side effects. In recent years, nanoparticle drug carrier systems have been developed to increase therapeutic efficiency and decrease side effects. These systems vary according to the agent they are prepared from and according to method of preparation. Polymeric nanoparticles, dendrimers, liposomes, polymeric micelles, solid lipid nanoparticles, lipid nanostructured systems are the most frequently used systems in this field. Polymeric nanoparticles are colloidal drug carrier systems that are prepared from generally natural or synthetic polymers whose size varies between 10-1000 nm. The size and surface properties of these systems are changed, and they can be made suitable for different aims. Therefore, not only active and passive targeting can be benefited from but also systems that provide controlled drug release can also be prepared. By means of these systems, not only controlled release of the active agent to the target area without losing the therapeutic effect of the active agent contained therein can be provided but also the spreading of the active agent to other organs and tissues of the body is preventing, thereby eliminating undesirable side effects. Nowadays there are over 50 medicines used clinically, for which licenses have been obtained and that have been developed using nanotechnological approaches, particularly Abraxane® and Venofer®. Moreover two of the 10 drugs that were best-selling in USA in 2013 were drugs that have been prepared with these principles (Copaxone® and Neulasta®). Nanodrugs and nanosimilar drugs that are equivalents of these are also approved and are sold in the market.

Studies related to nanoparticle drug carrier systems comprising cannabinoids are quite new and very limited number of studies is available in literature. Anandamide (AEA) loaded Polycaprolactone (PCL) nanoparticles, poly-lactic-co-glycolic acid (PLGA) nanoparticles loaded with CB13 that are synthetic cannabis-derivatives are available in the prior art. In a study carried out with CB13 which is a cannabinoid derivative, the optimization, characterization and in vitro viability analysis of nanoparticles have been carried out. As a result of the cell culture studies carried out, the cell viability in NIH3T3 (mouse embryonic fibroblast cell line), HEK293T (human embryonic kidney epithelial cell line) and Caco-2 (colorectal adenocarcinoma cell line) cells have been calculated to be close to 100%. It has been emphasized that a liposomal system that is nontoxic and is suitable to intestinal conditions comprising CB13 has been developed. It is also known that different cannabinoid derivatives (Tetrahydrocannabinol (THC), cannabidiol (CBD)) have cancer cell growth inhibiting properties. As a result of the studies carried out with PCL microparticles a 20-day release time has been obtained and it has been noted that apoptosis of the tumor increased when these particles are applied to mice with glioma locally for 5 days and that proliferation and vascularization was reduced. It has been emphasized that the system that has been developed as a result of this study can be used for cancer treatment via an alternative application route. The studies related to nanoparticle drug carrier systems comprising cannabinoids have summarized in Table 1. Accordingly, PCL nanoparticles and the ACPA carrier system subject to the invention is still not present in the prior art.

TABLE 1

| Cannabinoid | Release System | Working model | Target |
|---|---|---|---|
| AEA | PCL nanoparticle | in vitro | increase of cell |
| | | characterization | membrane |
| | | and release | permeability |
| | | | of AEA |
| CB13 | PGLA nanoparticle | in vitro | Oral application |
| | | characterization | in neuropathic |
| | | and release | pain treatment |
| CB13 | Lipid nanoparticle | in vitro | Oral application |
| | | characterization | |
| | | and release | |
| THC, CBD | Polycaprolactone | in vitro | Transdermal |
| | microparticles | characterization | application in |
| | | and release | cancer treatment |
| | | in vivo (mouse) | |
| | | cancer treatment | |
| SR141716 | Lipid nanoparticle | in vivo (rat) | Intranasal usage |
| SR141716, | Lipid nanoparticle | in vitro | Intranasal usage |
| AM251 | | characterization | |
| | | and release | |

Polycaprolactone (PCL) is a synthetic polymer commonly used in the preparation of polymeric nanoparticle drug delivery systems. The bioparticulate, semi-crystalline and aliphatic polymer approved by the American Food and Drug Administration (FDA) for implant biomaterial use in human biomedical applications is an important advantage compared to other synthetic polymers that are used for this purpose. As PCL is suitable for chemical modifications and as it can be copolymerized with other polymers, this allows PCL to be able to be used in the preparation of nanoparticle systems for different aims. As the degradation rate thereof is slow, it enables the preparation of systems that carry out controlled drug release and the preparation of implants that shall remain in the body for a long period of time. Some of the studies carried out with PCL nanoparticles in literature have been given in Table 2. As it has been specified in Table 2, several studies are available that are related to PCL nanoparticles in literature and nanoprecipitation has been selected as the preparation method in most of these studies.

TABLE 2

| | | Preparation | |
|---|---|---|---|
| Carrier | Active Agent | Method | Target Disease |
| PCL | Griseofulvin | Nanoprecipitation | Dermatomycosis |
| nanosphere | | | treatment |
| PCL | | | |
| nanocapsule | | | |
| PCL | Aripiprazole | Nanoprecipitation | Treatment of |
| nanoparticle | | | schizophrenia |
| PCL | Camptothecin | Nanoprecipitation | Treatment |
| nanoparticle | | | of glioma |
| PCL | Isradipine | Nanoprecipitation | Hypertension |
| nanoparticle | | | Treatment |
| PCL | 5-amino | Oil/Water | Inflammatory |
| nanoparticle | salicylic acid | Emulsion, | bowel disease |
| | | Nanoprecipitation | |
| PCL | Docetaxel | Nanoprecipitation | Breast cancer |
| nanoparticle | | | |
| PCL | Etoposide | Nanoprecipitation | Cancer |
| nanoparticle | | | Treatment |
| PCL | Ftorafur, | Interface polymer | Cancer |
| nanoparticle | Diclofenac | disposition | Treatment |
| | sodium | method | |
| PCL | Propranolol | Water/Oil/Water | — |
| nanoparticle | HCl | Emulsion | |
| PCL | AEA | Nanoprecipitation | — |
| nanoparticle | | | |

### SUMMARY

The primary aim of the invention is to identify a new therapeutic approach that creates a dose-dependent anti-proliferative effect by activating an existing mechanism in the body by investigating the effects of ACPA on non-small cell lung cancer.

By the usage of ACPA release system developed by means of the invention, controlled and a long-lasting effect is obtained in non-small cell lung cancer cells.

It is assumed by means of the invention that controlled release system of ACPA, which selectively targets the CB1 receptor that is widely distributed in non-small cell lung cancer cells, can provide a more effective treatment in comparison to other molecules used clinically, or that it can increase its potency in aggressive cases by being added into existing chemotherapy protocols.

An innovative drug candidate is presented with the invention, that can be administered alone or with other treatment protocols with controlled release of a new molecule having a molecularly effective and safe dose window for lung cancer that has a high mortality rate and no curative treatment. Thereby a method that can increase treatment efficiency and shorten treatment time clinically, in aggressive or advanced stage tumors can be developed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The figures that have been prepared in order to further explain the release system and the effect of cannabinoid 1 receptor (CB1) agonist arachidonoyl cyclopropylamide (ACPA) on non-small cell lung cancer subject to the invention are described below.

FIG. 1 shows the relative mRNA fold change of the CNR1 (CB1 receptor gene) and CNR2 (CB2 receptor gene) in non-small cell lung cancer cell lines A549, H1299, H358, H838, H1975 and SW-1573.

FIGS. 2A-2D show the WST-1 proliferation analysis results according to absorbance, (A450 nm-A630 nm) belonging to A549, H1299, H358 and H838 cells where ACPA is applied in the dose range of 10⁻¹²-10⁻⁶ M.

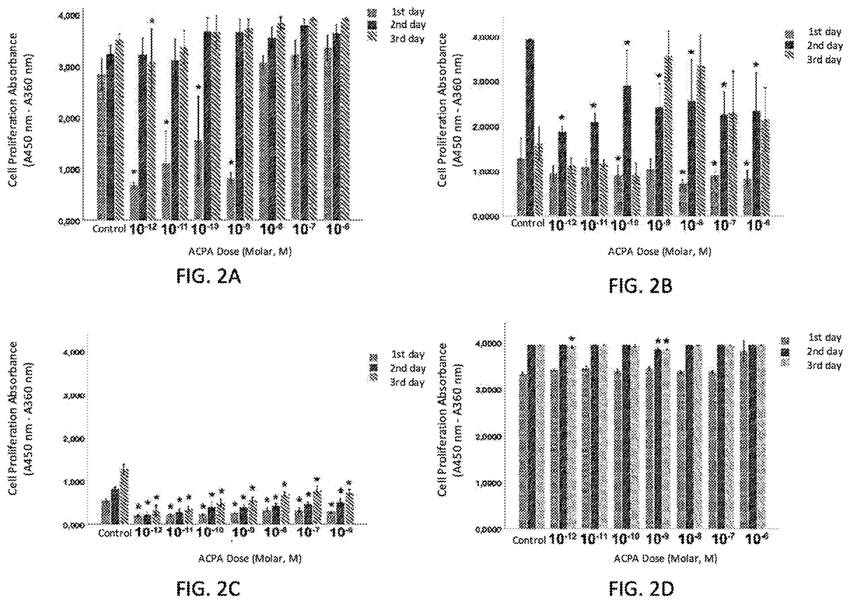

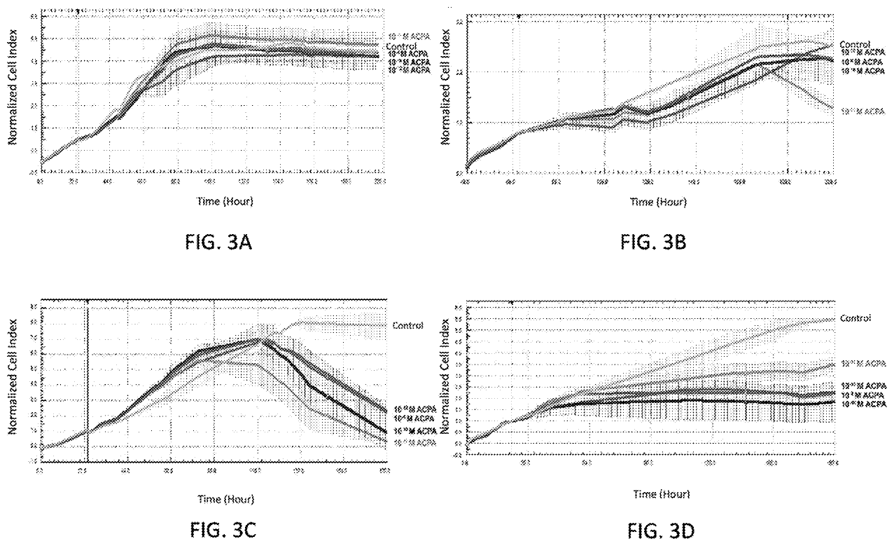

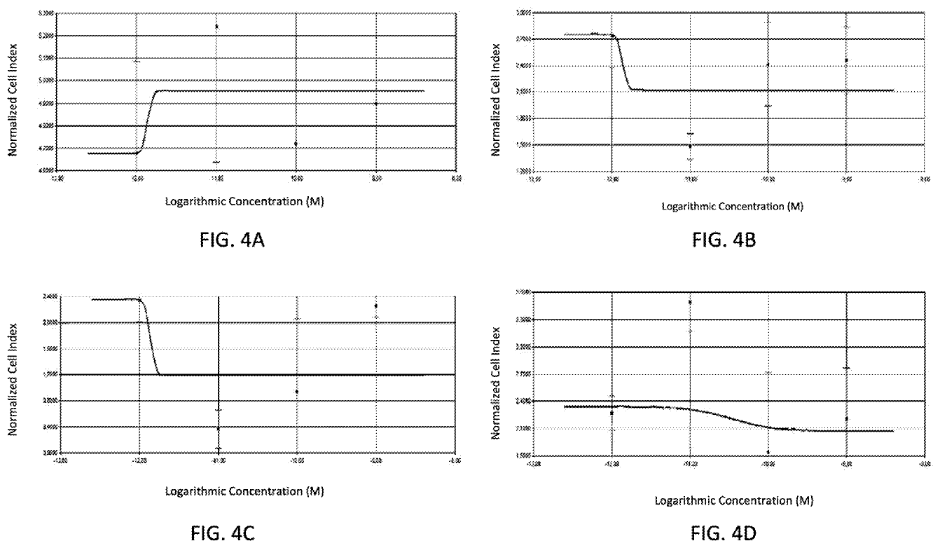

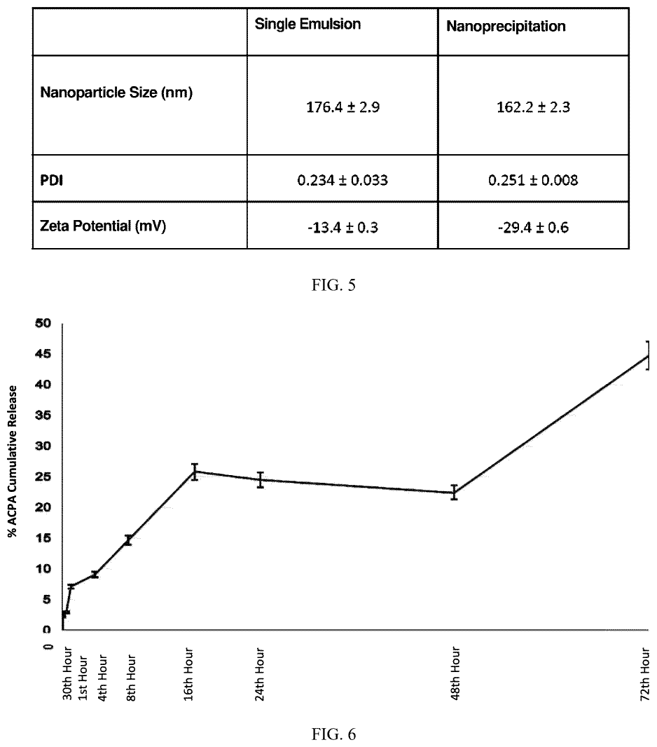

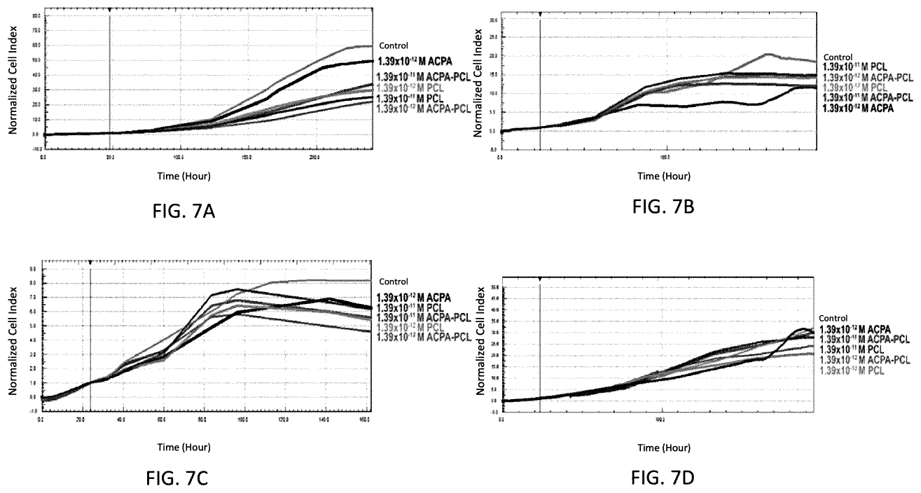

In FIG. 2A, the cell proliferation following doses of 10⁻¹², 10⁻¹¹, 10⁻¹⁰ M and 10⁻⁹ M of ACPA applied was seen to be reduced in the absorbance graph of A549 cells, at a statistically significant difference on day 1 in comparison to the control group (*p<0.05).

In FIG. 2B, significant antiproliferative effect has been observed in groups applied with ACPA in comparison to the control group on days 1, 2 and 3 according to the absorbance graph of H1299 cells (*p<0.05).

In FIG. 2C, the groups have exhibited antiproliferative effect in comparison to the control group in all doses on day 2, according to the absorbance graph of H358 cells (*p<0.05).

In FIG. 2D, the groups have exhibited antiproliferative effect in comparison to the control group at 10⁻⁹ M dose of ACPA on day 2, and 10⁻¹² and 10⁻⁹ M doses on day 3 according to the absorbance graph of H838 cells.

FIGS. 3A-3D show real time impedance-based proliferation analysis data of A549, H1299, H358 and H838 cells following ACPA application on day 1, 2 and 3.

In FIG. 3A, the normalized cell index against time (hour) of A549 cells is shown. After seeding the cells, the cell index has risen above 1.0 on the 24th hour.

In FIG. 3B, the normalized cell index against time (hour) of H1299 cells is shown. After seeding the cells, the cell index has risen above 1.0 on the 24th hour.

In FIG. 3C, the normalized cell index against time (hour) of H358 cells is shown. After seeding the cells, the cell index has risen above 1.0 on the 72nd hour.

In FIG. 3D, the normalized cell index against time (hour) of H838 cells is shown. After seeding the cells, the cell index has risen above 1.0 on the 24th hour.

FIGS. 4A-4D show the graph of the half maximal inhibitory concentration (IC50) of ACPA for A549, H1299, H358 and H838 cells.

In FIG. 4A, the normalized cell index against the logarithmic ACPA concentration (M) of A549 cells is shown. Accordingly, IC50 has been calculated as 1.39×10⁻¹² M for ACPA.

In FIG. 4B, the normalized cell index against the logarithmic ACPA concentration (M) of H1299 cells is shown. Accordingly, IC50 value has been calculated as 1.39×10⁻¹² M for ACPA.

In FIG. 4C, the normalized cell index against the logarithmic ACPA concentration (M) of H358 cells is shown. Accordingly, IC50 has been calculated as 1.38×10⁻¹² M for ACPA.

In FIG. 4D, the normalized cell index against the logarithmic ACPA concentration (M) of H838 cells is shown. Accordingly, IC50 value has been calculated as 3.47×10⁻¹¹ M for ACPA.

FIG. 5 shows the sizes of the prepared ACPA loaded PCL nanoparticles, polydispersity index (PDI) and zeta potential data.

FIG. 6 shows the controlled release graph of ACPA on the 30th minute, and hours, 1, 4, 6, 8, 16, 24, 48 and 72.

FIGS. 7A-7D show determination of real time impedance based antiproliferative effect of ACPA-PCL controlled release system in non-small cell lung cancer (NSCLC) cells, A549 (FIG. 7A), H1299 (FIG. 7B), H358 (FIG. 7A), H838 (FIG. 7D).

### DETAILED DESCRIPTION OF THE EMBODIMENTS

By means of the invention, the efficient and safe dose range of endogenous or synthetic cannabinoids on non-small cell lung adenocarcinoma cell lines that are frequently observed in society under prospective randomized, in vitro conditions were determined; the antiproliferative and proapoptotic effect mechanism within this dose window was examined and the antiproliferative effect it has on cells were examined by developing the ACPA-PCL controlled release system.

1. Evaluation of the dose- and time-dependent antiproliferative and proapoptotic effects of ACPA on A549, H1299, H358, H838, H1975 and SW-1573 non-small cell lung adenocarcinoma cell lines expressing CB receptors in vitro conditions

1.1. Evaluation of Cannabinoid CB1 and CB2 Receptor Expressions on Cell Lines by qRT-PCR

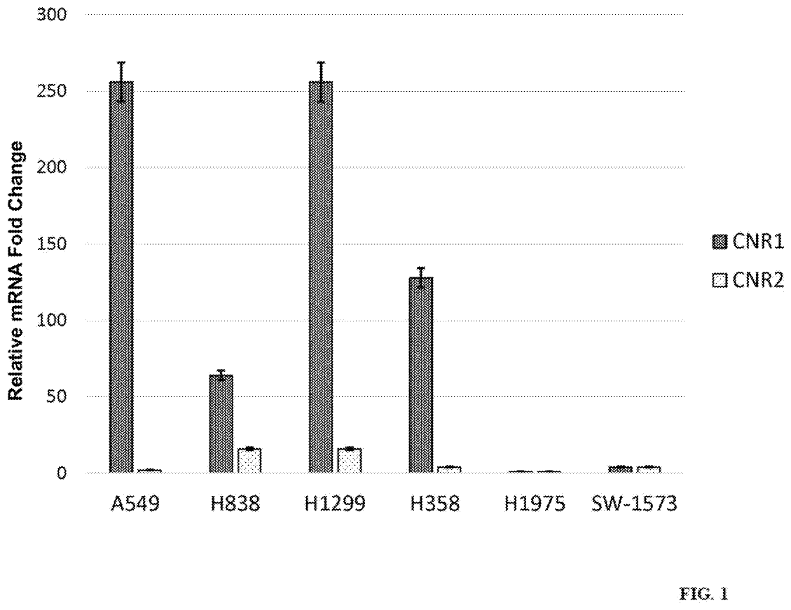

The CNR1 (CB1 receptor gene) and CNR2 (CB2 receptor gene) expression analysis on non-small cell lung cancer cell lines A549, H1299, H358, H838, H1975 and SW-1573 has been carried out with the qRT-PCR method. Accordingly, high levels of relative mRNA expression have been determined in A549, H1299, H838 and H358 cells. The graph belonging to the expression levels of CB1 and CB2 receptors have been given in FIG. 1.

### 1.2. Evaluation of the Dose- and Time-Dependent Effect of ACPA to Cancer Cell Proliferation by WST-1 Technique

ACPA has been applied to A549, H1299, H358 and H838 cells that have been determined to express the highest CB 1 receptor gene between non-small cell lung cancer cell lines at a dose range of 10⁻¹²-10⁻⁶ M. At doses of 10⁻¹², 10⁻¹¹, 10⁻¹⁰ M and 10⁻⁹ M of ACPA the proliferation has been statistically significantly reduced in A549 cells on the 1st day in comparison to the control group (*p<0.05). On the second and 3rd days antiproliferative effect was not observed at doses applied with ACPA. Antiproliferative effect has been observed on H1299 cells, in all doses of ACPA applied on days 1, 2 and 3 (*p<0.05). The antiproliferative effect of ACPA was observed on H358 cells on day 1, at doses of 10⁻¹⁰, 10⁻⁸, 10⁻⁷ and 10⁻⁶ M (*p<0.05). Antiproliferative effect was seen on H358 cells at all doses on the 2nd day of application of ACPA (*p<0.05).

ACPA has reduced the proliferation of H838 cells statistically significantly at doses of 10⁻⁹ M on the 2nd day and at doses of 10⁻⁹ and 10⁻¹² M on the 3rd day (*p<0.05) (FIG. 2A-2D).

1.3. Evaluation of the Effect of ACPA against Cancer Cell Proliferation by Real Time Impedance-Based Proliferation Analysis

ACPA has been found to be efficient between the dose range of 10⁻¹²-10⁻⁹ M on the 4 cell lines (A549, H1299, H358 and H838) it has been applied to, during WST-1 analysis. ACPA has been applied between this dose range (10⁻⁹, 10⁻¹⁰, 10⁻¹¹ and 10⁻¹² M) during the real time impedance-based proliferation analysis. After the cells are seeded when the cell indices reach 1.0, ACPA dose range has been applied. The graph has been normalized from the point where the cell indices reached 1.0 and ACPA application has been carried out at different doses. ACPA, has not exhibited a significant change in the proliferation of A549 cells (FIG. 3A). ACPA has reduced the proliferation of H1299 (FIG. 3B), H358 (FIG. 3C) and H838 (FIG. 3D) cells depending on time at the dose ranges applied. After ACPA has been added at the determined dose range, it has led to reduction in the proliferation of H1299 cells as of the 66th hour. As of the 66th hour 10⁻¹¹ M dose, after the 86th hour, 10⁻¹², 10⁻¹⁰ M and 10⁻⁹ M doses of ACPA has significantly reduced the proliferation of cells (p<0.05). The cell indices of the control group as of the 86th hour, has been found to be significantly excessive according to cell indices belonging to the ACPA dose range (p<0.05). The strongest antiproliferative effect of ACPA on the H358 cells has been observed following the 48th hour after the drug has been added. The cell index in the control group, has been found to be statistically significant when compared with all ACPA applied doses (p<0.05). The strongest antiproliferative effect of ACPA on the H358 cells has been observed following the 106th hour at the dose of 10⁻¹¹ M. After ACPA has been applied it has led to reduction in the proliferation of H838 cells as of the 30th hour. ACPA doses following the 30th hour, has significantly reduced the proliferation of cells in comparison to the control group (p<0.05). The cell indices of the control group as of the 40th hour, has been found to be significantly higher according to cell indices belonging to the ACPA dose range (p<0.05). The antiproliferative IC50 (effective dose that kills 50% of cells) value of ACPA effective on A549 cells has been calculated as 1.39×10⁻¹² M (FIG. 4A). The antiproliferative IC50 value of ACPA that is effective on H1299 cells has been calculated as 1.39×10⁻¹² M (FIG. 4B). The antiproliferative IC50 value of ACPA that is effective on H358 cells has been calculated as 1.38×10⁻¹² M (FIG. 4C). The antiproliferative IC50 value of ACPA that is effective on H838 cells has been calculated as 3.47×10⁻¹¹ M (FIG. 4D).

Within the scope of the research, the efficient dose and time of the CB1 receptor agonist ACPA has been calculated using the WST-1 and real time impedance-based proliferation analysis method on the examined 4 different non-small cell lung cancer lines. Accordingly, it has been noted that the effect of ACPA was seen on the 48th hour on A549, H1299 and H358 cells at the dose of 1.39×10⁻¹² M and on H838 cells at the dose of 3.47×10⁻¹¹ M. As a result, it has been determined that the efficient dose range was common for all cell lines and the efficient range was 10⁻¹¹ M-10⁻¹² M. Resulting dose range: 10⁻¹²-10⁻⁶ and effective dose range has been determined as 10⁻¹¹ M-10⁻¹² M.

2. Preparation of the Controlled Release System for CB1 Agonist ACPA that has been Determined to be Efficient

### 2.1. Preparation of the Nanoparticle Formulations for the Prepared Controlled Release System

The synthetic polycaprolactone (PCL) polymer has been used, which is a hydrophobic polymer for the preparation of ACPA loaded polymeric nanoparticle formulations and precipitation and single emulsion methods that are used frequently for this purpose in literature has been tried and the most suitable and simple preparation method has been selected. Accordingly, a dichloromethane: ethanol (DCM: EtOH) (9:1; v/v) mixture comprising 0.1% (w/v) PCL and 0.01% (w/v) ACPA (100 μg) has been prepared for the single emulsion method. Following this, the mixture has been placed into a mixer at 550 rpm the ultra-pure water comprising 1% (w/v) Pluronic F68® has been added to the mixture by means of an injector. The final mixture was mixed at a speed of 550 rpm for 30 minutes. Following the mixing period, in order for the organic solvents to be removed from the mixture, the mixture has been left inside a rotary evaporator for 30 minutes and after the nanoparticles were passed through 45 μm pore size filter an aqueous dispersion has been obtained. Acetonitrile: ethanol (9:1; v/v) mixture comprising 0.1% (w/v) PCL and 0.01% (w/v) ACPA has been prepared for the precipitation method. Following this, the mixture has been placed into a mixer at 550 rpm the ultra-pure water comprising 1% (w/v) Pluronic F68R has been added to the mixture with an injector. The final mixture was mixed at 550 rpm for 30 minutes. After the mixture was kept in a rotary evaporator for 30 minutes, the nanoparticle dispersions are passed through 45 μm pore size filters and the free ACPA was removed. Following the characterization of the nanoparticle, the nanoparticles have been prepared with a single method.

After the nanoparticle dispersions were prepared the nanoparticles were lyophilized for 48 hours and they have been turned into powder form. The powder nanoparticles are dissolved in dichloromethane (DCM) and this solution has been added into ethanol in order for the nanoparticles to be completely dissolved and for the loaded drug to be released. The release profile assays have been carried out by means of this process.

### 2.2. In Vitro Characterization of the Prepared Nanoparticle Formulation

The characterization studies of the prepared nanoparticle formulations have been carried out in vitro and the polydispersity index of the nanoparticles the particle size, surface load, drug loading efficiency and drug release profile have been examined. The most suitable nanoparticle formulation for the target region has been determined to be used in cell culturing studies.

The particle size and the polydispersity index (PDI) of the nanoparticles have been carried out via the dynamic light scattering technique and capillary zeta forces. Zetasizer Nano ZS device has been used to determine the zeta potential and the surface load of the nanoparticles. 0.9 ml samples have been taken in order to determine PDI and the particle sizes of the prepared nanoparticle dispersions and they have been placed into Zeta tanks. Designation has been carried out at an angle of 173° with n=3 repeats at a room temperature of 25° C. for particle size designation. Surface load measurements were carried out at an angle of 12.8° and at room temperature simultaneously with particle size determination and were given in mV. The APCA-PCL nanoparticle sizes that have been prepared with the single emulsion method have been obtained as 176.4±2.9; PDI 0.234±0.033 and Zeta potential: −13.4±0.3 mV. The sizes of the APCA-PCL nanoparticles that have been obtained by means of the nanoprecipitation (precipitation) method, have been obtained as 162.2±2.3; PDI 0.251±0.008 and Zeta potential: −29.4±0.6 mV (FIG. 5).

LC-MS/MS analytical amount determination method was used in the measurement of drug loading capacity and preparation of the drug release profile. For drug loading efficiency, nanoparticle samples were measured with n=6 repeats. Dialysis membrane method was used to determine the drug release profile. After the drug loaded nanoparticle formulations are prepared, the nanoparticle dispersions are passed through a filter and free ACPA is removed and following this a sample was taken and was placed inside a dialysis membrane (MWCO=14.000 Da). Determination of the amount of ACPA was carried out by the LC-MS/MS method by taking a 2 ml sample with n=3 repeats within different time ranges (0 minutes, 30th minute and on the 1st, 4th, 8th, 16th, 24th, 48th and 72nd hours) by having this dialysis being placed inside a pH 7.4 phosphate buffer such that the final volume is 25 ml and such that it meets the membrane sink conditions. The release medium has been kept at a constant degree of 37° C. during the release assays and the agitation speed has been determined to be 100 rpm. The loading efficiency has been given as encapsulation efficiency (% EE) and has been calculated according to the below mentioned formula.

$$\% Encapsulation Efficiency ( \% EE ) = \frac{Measured DOC amount ( μg )}{Initial DOC amount ( μg )} \times 100$$

Accordingly, 2.2±2.3% of ACPA loaded at 0 minute is released to the medium. 2.9±3.0% of ACPA is released on the 30th minute, 7.1±7.6% is relaxed on the 60th minute, 9.1±4.1% is released on the 240th minute, 14.6±6.7% is released on the 480th minute. 25.8±7.4% of ACPA is released on the 960th minute, 24.5±5.2% is released on the 1440th minute, 22.4±10.8% is released on the 2880 minute and 44.7±9.9% is released on the 4320th minute. At the end of the release period ACPA has released 44.7% of the loaded amount into the medium (FIG. 6). The loading efficiency has been calculated as encapsulation efficiency (% EE). ACPA, has been loaded to PCL nanoparticles, at the ratio of 39.9±14.7% (Table 3).

TABLE 3

| Average | | | |
|---|---|---|---|
| Loaded | | | |
| ACPA | Standard | % | % |
| Amount | Deviation | Loading | Standard |
| (μg) | (μg) | Efficiency | Deviation |
| 39.9 | 14.7 | 39.9 | 14.7 |

3. Impedance Based and Real Time Evaluation of the Effect of the Prepared Drug Release System on the Proliferation of Non-Small Cell Lung Cancer

The prepared ACPA-PCL nanoparticles have been diluted inside the medium such that 1.39×10⁻¹² M ACPA which is an IC50 dose is released. After the drug is added, following the 96th hour, all of the doses have shown antiproliferative effect on the A549 cell line (p<0.05). PCL nanoparticles releasing ACPA has reduced the proliferation of A549 cells as efficiently as possible (FIG. 7A). After the PCL nanoparticles that release 1.39×10⁻¹² M ACPA and 1.39×10⁻¹¹ M ACPA dose are added, the proliferation of H1299 cells have been significantly reduced relative to control on the 24th hour (p<0.05). After the drug is added, on the 48 and 72nd hours, significant reduction has been observed in comparison to the control group in groups of PCL nanoparticles that release 1.39×10⁻¹² M ACPA dose, blank PCL nanoparticles that have been diluted with the same volume as 1.39×10⁻¹² M ACPA and the groups applied with 1.39×10⁻¹² M ACPA (p<0.05). In the H1299 cells the control group was found to be statistically significantly excessive in comparison to all other groups as of the 96th hour (p<0.05) (FIG. 7B). PCL nanoparticles that release 1.39×10⁻¹² M ACPA dose and 1.39×10⁻¹¹ M ACPA dose have not exhibited the statistically significant antiproliferative effect on the H358 cells until the 140′th hour (FIG. 7A). Following the 140th hour, control group was found to be statistically significantly excessive in comparison to assay groups (p<0.05) (FIG. 7C). The PCL nanoparticles that release 1.39×10⁻¹² M and 1.39×10⁻¹¹ M ACPA dose have not exhibited the statistically significant antiproliferative effect on the H838 cells for 195 hours. It has been determined that the blank PCL nanoparticles that were not loaded with 1.39×10⁻¹² M ACPA and drugs, showed statistically significant reduction on the 190th hour on H838 cells in comparison to the control group (p<0.05) (FIG. 7D).

The invention is related to a drug comprising cannabinoid 1 receptor agonist arachidonoyl cyclopropylamide (ACPA) to be used in the treatment of non-small cell lung cancer.

The drug that is used in treating non-small cell lung cancer can comprise 10⁻¹²-10⁻⁶ M cannabinoid 1 receptor agonist arachidonoyl cyclopropylamide (ACPA).

The drug that is used in treating non-small cell lung cancer can comprise 10⁻¹¹ M-10⁻¹²M cannabinoid 1 receptor agonist arachidonoyl cyclopropylamide (ACPA).

The drug containing cannabinoid 1 receptor agonist arachidonoyl cyclopropylamide (ACPA) can be effective on a plurality of non-small cell lung cancer types.

The drug that is used in treating non-small cell lung cancer can comprise at least a pharmaceutically acceptable carrier besides cannabinoid 1 receptor agonist arachidonoyl cyclopropylamide (ACPA).

The drug that is used in treating non-small cell lung cancer comprises at least a pharmaceutically acceptable carrier besides cannabinoid 1 receptor agonist arachidonoyl cyclopropylamide (ACPA) at dose range of 10⁻¹²-10⁻⁶ M.

The drug that is used in treating non-small cell lung cancer comprises at least a pharmaceutically acceptable carrier besides cannabinoid 1 receptor agonist arachidonoyl cyclopropylamide (ACPA) at dose range of 10⁻¹¹-10⁻¹² M.

The drug containing at least a pharmaceutically acceptable carrier besides cannabinoid 1 receptor agonist arachidonoyl cyclopropylamide (ACPA) can be effective on a plurality of non-small cell lung cancer types.

The at least a pharmaceutically acceptable carrier besides the cannabinoid 1 receptor agonist arachidonoyl cyclopropylamide (ACPA) contained in the drug is a polymeric nanoparticle.

In an embodiment of the invention the polymeric nanoparticle that is at least a pharmaceutically acceptable carrier besides the cannabinoid 1 receptor agonist arachidonoyl cyclopropylamide (ACPA) contained in the drug, comprises polycaprolactone (PCL).

The release of the drug containing at least a pharmaceutically acceptable carrier besides the cannabinoid 1 receptor agonist arachidonoyl cyclopropylamide (ACPA) into the body is carried out by loading arachidonoyl cyclopropylamide (ACPA) into the carrier.

The formulation formed with the at least a pharmaceutically acceptable carrier besides the cannabinoid 1 receptor agonist arachidonoyl cyclopropylamide (ACPA) is in the form of a tablet, capsule, powder, granule, pill or spray (inhaler).

1. A method of treating non-small cell lung cancer, comprising a step of administering a drug to a subject, wherein the drug comprises cannabinoid 1 receptor agonist arachidonoyl cyclopropylamide (ACPA), a dose of the ACPA is in a range of 10⁻¹²-10⁻⁹ M and the ACPA is effective on a plurality of non-small cell lung cancer types.

2. The method according to claim 1, wherein the drug further comprises at least a pharmaceutically acceptable carrier, wherein the pharmaceutically acceptable carrier is a polymeric nanoparticle comprising polycaprolactone (PCL).

3. The method according to claim 2, wherein the ACPA is released into a body after being loaded into the pharmaceutically acceptable carrier.

4. The method according to claim 2, wherein the drug is in a form of a tablet, a capsule, a powder, a granule, a pill or an inhaler.

5. The method according to claim 2, wherein the drug is formulated as a lyophilized particle or nanoparticle dispersion having a shell, comprising PCL containing ACPA.

6. The method according to claim 5, wherein the drug is formulated as a nanoparticle dispersion and PCL and ACPA are present as 1:10 ratio (0.01% (w/v) ACPA:0.1% (w/v) PCL) in acetonitrile: ethanol (9:1; v/v) mixture by nanoprecipitation method.

7. The method according to claim 6, wherein the nanoparticle comprises ultra pure water and a non-ionic surfactant.

8. The method according to claim 7, wherein the nanoparticle has a non-ionic surfactant of 1% (w/v) Pluronic F68°.

9. The method according to claim 5, wherein the nanoparticle dispersion is passed through a 45 μm pore size filter.

10. The method according to claim 9, wherein the nanoparticle dispersion is lyophilized for 48 hours to obtain a nanoparticle powder product.
